(19) United States
(12) Patent Application Publication
(10) Pub. No.: US 2009/0105324 A1
(43) Pub. Date: Apr. 23, 2009

(54) IMIDAZOLE DERIVATIVES FOR USE AS EDG-1 ANTAGONISTS

(75) Inventors: Gurmit Grewal, Waltham, MA (US); Vibha Oza, Waltham, MA (US); Jamal Carlos Saeh, Waltham, MA (US); Qibin Su, Waltham, MA (US)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 12/297,799

(22) PCT Filed: Apr. 20, 2007
(86) PCT No.: PCT/GB07/01447

§ 371 Date: Oct. 20, 2008

Related U.S. Application Data
(60) Provisional application No. 60/745,296, filed on Apr. 21, 2006.

(51) Int. Cl. A61K 31/4164; C07D 233/61; A61P 35/00; A61P 35/04

(52) U.S. Cl. 514/400; 548/340.1

(57) ABSTRACT

The invention relates to chemical compounds of formula (I): or pharmaceutically acceptable salts thereof, which possess Edg-1 antagonistic activity and are accordingly useful for their anti cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm blooded animal such as man.

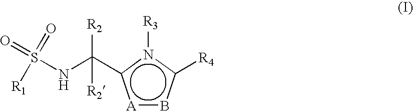

### BACKGROUND OF THE INVENTION

EDG (endothelial differentiation gene) receptors belong to a family of closely related, lipid activated G-protein coupled receptors. EDG-1, EDG-3, EDG-5, EDG-6, and EDG-8 (also known as S1P1, S1P3, S1P2, S1P4, and S1P5) are identified as receptors specific for sphingosine-1-phosphate (SIP). EDG2, EDG4, and EDG7 (known also as LPA1, LPA2, and LPA3, respectively) are receptors specific for lysophosphatidic (LPA). Among the SIP receptor isotypes, EDG-1, EDG-3 and EDG-5 are widely expressed in various tissues, whereas the expression of EDG-6 is confined largely to lymphoid tissues and platelets, and that of EDG-8 to the central nervous system.

EDG receptors are responsible for signal transduction and are thought to play an important role in cell processes involving cell development, proliferation, maintenance, migration, differentiation, plasticity and apoptosis. Certain EDG receptors are associated with diseases mediated by the de novo or deregulated formation of vessels—for example, for diseases caused by ocular neovascularisation, especially retinopathies (diabetic retinopathy, age-related macular degeneration); psoriasis; haemangioblastomas such as “strawberry-marks”; various inflammatory diseases, such as arthritis, especially rheumatoid arthritis, arterial atherosclerosis and atherosclerosis occurring after transplants, endometriosis or chronic asthma; and tumor diseases. EDG receptors are also associated with various inflammatory diseases, such as arthritis, especially rheumatoid arthritis, arterial atherosclerosis and atherosclerosis occurring after transplants, endometriosis or chronic asthma; and, especially, tumor diseases or by lymphocyte interactions, for example, in transplantation rejection, autoimmune diseases, inflammatory diseases, infectious diseases and cancer. An alteration in EDG receptor activity contributes to the pathology and/or symptomology of these diseases. Accordingly, molecules that themselves alter the activity of EDG receptors are useful as therapeutic agents in the treatment of such diseases.

### SUMMARY OF THE INVENTION

In accordance with the present invention, the applicants have hereby discovered novel compounds that are Edg-1 antagonists. These compounds of the present invention provide a method for treating a variety of angiogenesis-related diseases that may be characterized by any abnormal, undesirable or pathological angiogenesis, for example, tumor-related angiogenesis. The compounds may be used to produce an anti-cancer effect mediated alone or in part by antagonism of Edg-1. Such a compound of the invention is expected to possess a wide range of activity in angiogenesis-related diseases including, but not limited to, non-solid tumors such as leukemia, multiple myeloma, hematologic malignancies or lymphoma, and also solid tumors and their metastases such as melanoma, non-small cell lung cancer, glioma, hepatocellular (liver) carcinoma, glioblastoma, carcinoma of the thyroid, bile duct, bone, gastric, brain/CNS, head and neck, hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid tumors. The compounds of the invention are accordingly useful for their anti-angiogenic (such as anti-cancer) activity and are therefore useful in methods of treatment of the human or animal body.

The invention also relates to processes for the manufacture of said compounds, to pharmaceutical compositions containing them and to their use in the manufacture of medicaments for use in the production of an anti-cancer effect, for example an anti-proliferative effect, in warm-blooded animals such as man.

The present invention includes pharmaceutically acceptable salts of such compounds. Also in accordance with the present invention applicants provide pharmaceutical compositions and a method to use such compounds in the treatment of cancer.

### DETAILED DESCRIPTION OF THE INVENTION

Accordingly, the present invention provides a compound of formula I

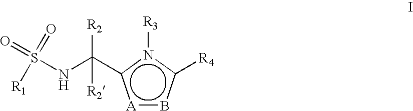

or a pharmaceutically acceptable salt thereof, wherein:

A and B are each independently N or CR_{b}, provided that A and B are not both N;

R_{b} at each occurrence is H, halo, (C₁-C₆)alkyl, cyano, —C(O)—(C₁-C₆)alkyl, —CO₂(C₁-C₆)allyl, C(O)—NR′R″, wherein R′ and R″ are each independently at each occurrence H or (C₁-C₆)alkyl or X—R_{c}; —CO₂H, —SO₂NHR;

R₁ is aryl, heteroaryl, (C₁-C₆)alkyl, aralkyl, heterocycloalkyl, or heteroaralkyl;

R₂ and R_{2′} are each independently H, (C₁-C₆)alkyl, aryl, heteroaryl, aralkyl, or heteroaralkyl, or taken together with the carbon to which they are attached from C═O;

R₃ is (C₁-C₆)alkyl, (C₃-C₆)cycloalkyl(C₁-C₆)alkyl, heterocycloalkyl, aralkyl, (C₂-C₆)alkenyl, (C₂-C₆)alkynyl, or heteroaralkyl, or X—R_{c};

R₄ is (C₁-C₆)alkyl, (C₃-C₆)cycloalkyl(C₁-C₆)alkyl, heterocycloalkyl, aralkyl, (C₂-C₆)alkenyl, (C₂-C₆)alkynyl, or heteroaralkyl, or X—R_{c};

X is S, O, or NR_{d};

R_{c} is H or (C₁-C₆)alkyl;

R_{d} is H, (C₁-C₆)alkyl, aryl, heteroaryl, heterocyclo, (C₂-C₆)alkenyl, (C₂-C₆)alkynyl, aralkyl, heteroaralkyl, (C₃-C₆)cycloalkyl(C₁-C₆)alkyl, heterocycloalkyl(C₁-C₆)alkyl, acyl, acyloxy, acylamino, or (C₁-C₆)alkoxycarbonyl(C₁-C₆)alkyl, or cyano; and

each R₁, R₂, R_{2′}, R₃, Rₐ, R_{b}, R_{c}, and R_{d} may be optionally substituted on carbon by azido, halo, nitro, cyano, hydroxy, trifluoromethoxy, NR′R″, —CO₂H, C(O)—(C₁-C₆)alkyl, —CO₂(C₁-C₆)alkyl, —C(O)—NR′R″, S(C₁-C₆), SOₚ(C₁-C₆)alkyl, SOₚNH(C₁-C₆)alkyl, SOₚNR′R″ (C₂-C₆)alkenyl, (C₂-C₆)alkynyl, or (C₁-C₆)alkoxy, wherein R′ and R″ are each independently hydrogen, (C₁-C₆)alkyl, (C₃-C₆)cycloalkyl, (C₃-C₆)cycloalkyl(C₁-C₆)alkyl, or aryl.

The invention is also directed to compounds of formula I or a pharmaceutically acceptable salt thereof, wherein:

A and B are each independently N, NRₐ, or CR_{b}, provided that A and B are not both N;

Rₐ independently at each occurrence is H, (C₁-C₆)alkyl, C(O)—(C₁-C₆)alkyl, C(O)—NR′R″, CO₂(C₁-C₆)alkyl;

R_{b} at each occurrence is H, halo, (C₁-C₆)alkyl, cyano, —C(O)—(C₁-C₆)alkyl, —CO₂(C₁-C₆)alkyl, C(O)—NR′R″, wherein R′ and R″ are each independently at each occurrence H or (C₁-C₆)alkyl or X—R_{c}; —CO₂H, —SO₂NHR;

R₁ is optionally substituted aryl, heteroaryl, (C₁-C₆)alkyl, aralkyl, heterocycloalkyl, or heteroaralkyl;

R₂ and R_{2′} are each independently H, (C₁-C₆)alkyl, aryl, heteroaryl, aralkyl, or heteroaralkyl, or taken together with the carbon to which they are attached from C═O;

R₃ is (C₁-C₆)alkyl, (C₃-C₆)cycloalkyl(C₁-C₆)allyl, heterocycloalkyl, aralkyl, (C₂-C₆)alkenyl, (C₂-C₆)alkanyl, or heteroaralkyl, or X—R_{c};

R₄ is (C₁-C₆)alkyl, (C₃-C₆)cycloalkyl(C₁-C₆)alkyl, heterocycloalkyl, aralkyl, (C₂-C₆)alkenyl, (C₂-C₆)alkynyl, or heteroaralkyl, or X—R_{c};

X is S, O, or NR_{d};

R_{c} is H or (C₁-C₆)alkyl;

R_{d} is H, (C₁-C₆)alkyl, aryl, heteroaryl, heterocyclo, (C₂-C₆)alkenyl, (C₂-C₆)alkynyl, aralkyl, heteroaralkyl, (C₃-C₆)cycloalkyl(C₁-C₆)alkyl, heterocycloalkyl(C₁-C₆)alkyl, acyl, acyloxy, acylamino, or (C₁-C₆)alkoxycarbonyl(C₁-C₆)alkyl, or cyano; and

each R₁, R₂, R_{2′}, R₃, Rₐ, R_{b}, R_{c}, and R_{d} may be optionally substituted on carbon by azido, halo, nitro, cyano, hydroxy, trifluoromethoxy, NR′R″, —CO₂H, C(O)—(C₁-C₆)alkyl, —CO₂(C₁-C₆)alkyl, —C(O)—NR′R″, S(C₁-C₆), SOₚ(C₁-C₆)alkyl, SOₚNH(C₁-C₆)alkyl, SOₚNR′R″ (C₂-C₆)alkenyl, (C₂-C₆)alkynyl, or (C₁-C₆)alkoxy, wherein R′ and R″ are each independently hydrogen, (C₁-C₆)alkyl, (C₃-C₆)cycloalkyl, (C₃-C₆)cycloalkyl(C₁-C₆)alkyl, or aryl.

The invention is also directed to compounds of formula I which are:

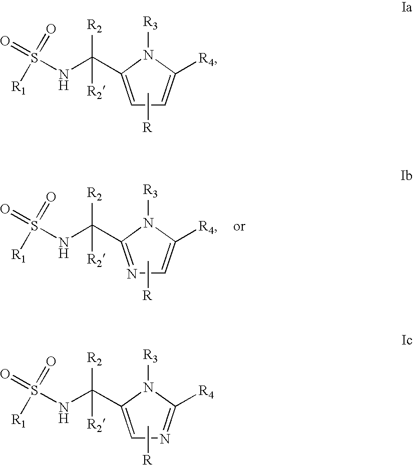

or a pharmaceutically acceptable salt thereof, wherein

R is H, halo, (C₁-C₆)alkyl, cyano, —C(O)—(C₁-C₆)alkyl, —CO₂(C₁-C₆)alkyl, C(O)—NR′R″, wherein R′ and R″ are each independently at each occurrence H or (C₁-C₆)alkyl or X—R_{c}; —CO₂H, —SO₂NHR; and

R₁, R₂, R_{2′}, R₃, and R₄ are as defined for a compound of formula I.

Unless otherwise stated, the following terms used in the specification and claims have the following meanings.

### DEFINITIONS

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, pentyl, and the like.

“Alkylene” means a linear saturated divalent hydrocarbon radical of one to six carbon atoms or a branched saturated divalent hydrocarbon radical of three to six carbon atoms, e.g., methylene, ethylene, propylene, 2-methylpropylene, pentylene, and the like.

“Alkenyl” means a linear monovalent hydrocarbon radical of two to six carbon atoms or a branched monovalent hydrocarbon radical of three to six carbon atoms, containing at least one double bond, e.g., ethenyl, propenyl, and the like.

“Alkynyl” means an alkyl group having one or more carbon-carbon triple bonds, e.g., ethynyl.

“Cycloalkyl” means a saturated monovalent cyclic hydrocarbon radical of three to six ring carbons, e.g., cyclopropyl, cyclohexyl, and the like.

“Aryl” means a monovalent monocyclic or bicyclic aromatic hydrocarbon radical of 6 to 10 ring atoms, and optionally substituted independently with one or more substituents, preferably one, two or three substituents selected from alkyl, haloalkyl, heteroalkyl, cycloalkyl, cycloalkylalkyl, halo, cyano, nitro, acyloxy, alkoxy, optionally substituted phenyl, heteroaryl, heteroaralkyl, amino, monosubstituted amino, disubstituted amino, acylamino, hydroxylamino, amidino, guanidino, cyanoguanidinyl, hydrazino, hydrazido, —OR [where R is hydrogen, alkyl, haloalkyl, alkenyl, cycloalkyl, cycloalkylalkyl, optionally substituted phenyl, heteroaryl or heteroaralkyl], —S(O)ₙR [where n is an integer from 0 to 2 and R is hydrogen, alkyl, haloalkyl, alkenyl, cycloalkyl, cycloalkylalkyl, optionally substituted phenyl, heteroaryl, heteroaralkyl, amino, mono or disubstituted amino], —NRSO₂R′ (where R is hydrogen or alkyl and R′ is alkyl, amino, monosubstituted or disubstituted amino) —C(O)R (where R is hydrogen, allyl, alkenyl, cycloalkyl, heteroalkyl, haloalkyl or optionally substituted phenyl), —COOR (where R is hydrogen, alkyl, optionally substituted phenyl, heteroaryl or heteroaralkyl), -(alkylene)-COOR (where R is hydrogen, alkyl, optionally substituted phenyl, heteroaryl or heteroaralkyl), methylenedioxy, 1,2-ethylenedioxy, —CONR′R″ or -(alkylene)CONR′R″ (where R′ and R″ are independently selected from hydrogen, alkyl, cycloalkyl, haloalkyl, cycloalkylalkyl, optionally substituted phenyl, heteroaryl and heteroaralkyl). More specifically the term aryl includes, but is not limited to, phenyl, 1-naphthyl, 2-naphthyl, and derivatives thereof.

“Aralkyl” means a radical —Rₐ—R_{b} where Rₐ is bound to R_{b} and Rₐ is an alkylene group and R_{b} is an aryl group as defined above e.g., benzyl, and the like.

“Heterocycle” or “heterocyclyl” means a saturated or partially unsaturated cyclic radical of 3 to 8 ring atoms in which one or two ring atoms are heteroatoms selected from NH, NRₐ as defined above, O, SO, OR SO₂.

“Heteroaryl” means an optionally substituted monovalent monocyclic radical of 5 or 6 ring atoms containing one, two, or three ring heteroatoms selected from N, O, or S, the remaining ring atoms being C. The term heteroaryl includes, but is not limited to pyridyl, pyrrolyl, thiophene, pyrazolyl, thiazolyl, imidazolyl, pyrimidinyl, thiadiazolyl, carbazolyl, and derivatives thereof.

“Heteroaralkyl” means a radical —Rₐ—R_{b} where Rₐ is bound to R_{b} and Rₐ is an alkylene group and R_{b} is a heteroaryl group as defined above e.g., pyridin-3-ylmethyl, 3-(benzofuran-2-yl)propyl, and the like.

“Optionally substituted” means that the group at issue is optionally substituted independently with one, two or three substituents selected from alkyl, haloalkyl, halo, nitro, cyano, —OR (where R is hydrogen or alkyl), —NRR′ (where R and R′ are independently of each other hydrogen or alkyl), —COOR (where R is hydrogen or alkyl) or —CONR′R″ (where R′ and R″ are independently selected from hydrogen or alkyl), or as otherwise provided.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylaime, piperidine, morpholine or tris-(2-hydroxyethyl)amine.

Some compounds of the formula I may have chiral centres and/or geometric isomeric centres (E- and Z-isomers), and it is to be understood that the invention encompasses all such optical, diastereoisomers and geometric isomers that possess Edg-1 antagonistic activity. The invention further relates to any and all tautomeric forms of the compounds of the formula I that possess Edg-1 antagonistic activity.

It is also to be understood that certain compounds of the formula I can exist in solvated as well as unsolvated forms such as, for example, hydrated forms. It is to be understood that the invention encompasses all such solvated forms that possess Edg-1 antagonistic activity.

Particular values of variable groups are as follows. Such values may be used where appropriate with any of the definitions, claims or embodiments defined hereinbefore or hereinafter unless specifically indicated otherwise.

A is N.

B is N.

A is CR_{b}.

B is CRₐ.

A is CR_{b} where R_{b} is H.

B is CR_{b} where R_{b} is H.

A is CR_{b} and B is N.

B is CR_{b} and A is N.

A is CH and B is N.

B is CH and A is N.

R₁ is aryl wherein aryl may be optionally substituted on carbon by azido, halo, nitro, cyano, hydroxy, trifluoromethoxy, NR′R″, —CO₂H, C(O)—(C₁-C₆)alkyl, —CO₂(C₁-C₆)allyl, —C(O)—NR′R″, S(C₁-C₆), SOₚ(C₁-C₆)alkyl, SOₚNH(C₁-C₆)alkyl, SOₚNR′R″ (C₂-C₆)alkenyl, (C₂-C₆)alkynyl, or (C₁-C₆)alkoxy, wherein R′ and R″ are each independently hydrogen, (C₁-C₆)alkyl, (C₃-C₆)cycloalkyl, (C₃-C₆)cycloalkyl(C₁-C₆)alkyl, or aryl.

R₁ is aryl wherein aryl may be optionally substituted by halo.

R₁ is chlorophenyl.

R₁ is p-chlorophenyl.

R₂ is H.

R₂ is H.

R₂ is aralkyl.

R_{2′} is aralkyl.

R₂ is phenylmethyl.

R_{2′} is phenylmethyl.

R₃ is (C₁-C₆)alkyl.

R₃ is methyl.

R₄ is (C₁-C₆)alkyl.

R₄ is methyl.

In another embodiment the invention is directed to a compound of formula I or pharmaceutically acceptable salt thereof wherein

A is N;

B is CR_{b};

R_{b} is selected from H, halo, (C₁-C₆)alkyl, cyano, —C(O)—(C₁-C₆)alkyl, —CO₂(C₁-C₆)alkyl, C(O)—NR′R″, wherein R′ and R″ are each independently at each occurrence H or (C₁-C₆)alkyl or X—R; —CO₂H, —SO₂NHR;

R₁ is aryl wherein aryl may be optionally substituted on carbon by azido, halo, nitro, cyano, hydroxy, trifluoromethoxy, NR′R″, —CO₂H, C(O)—(C₁-C₆)alkyl, —CO₂(C₁-C₆)alkyl, —C(O)—NR′R″, S(C₁-C₆), SOₚ(C₁-C₆)alkyl, SOₚNH(C₁-C₆)allyl, SOₚNR′R″ (C₂-C₆)alkenyl, (C₂-C₆)alkynyl, or (C₁-C₆)alkoxy, wherein R′ and R″ are each independently hydrogen, (C₁-C₆)alkyl, (C₃-C₆)cycloalkyl, (C₃-C₆)cycloalkyl(C₁-C₆)allyl, or aryl;

R₂ is selected from H and aralkyl;

R_{2′} is selected from H and aralkyl;

R₃ is (C₁-C₆)alkyl; and

R₄ is (C₁-C₆)alkyl.

In a further embodiment the invention is directed to a compound of formula I or pharmaceutically acceptable salt thereof wherein

A is N;

B is CH;

R₁ is phenyl wherein phenyl may be optionally substituted on carbon by halo;

R₂ is aralkyl;

R_{2′} is H;

R₃ is (C₁-C₆)alkyl; and

R₄ is (C₁-C₆)alkyl.

In a still further embodiment the invention is directed to the compound of Example 1 or a pharmaceutically acceptable salt thereof.

What is also provided is a process for the preparation of a compound of formula I as summarized in Scheme 1 infra.

Another aspect of the present invention provides a processes preparing compounds of formula I or a pharmaceutically acceptable salt thereof which process (wherein variable groups are, unless otherwise specified, as defined in formula I) comprises the following:

a) reacting a compound of formula (1 e)

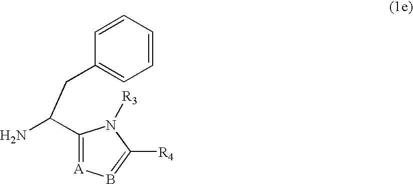

with a compound of formula (2)

R₁SO₂Cl (2)

and thereafter if necessary:
i) converting a compound of the formula I into another compound of the formula I;
ii) removing any protecting groups;
iii) forming a pharmaceutically acceptable salt.

### Preparation of Invention Compounds

Compounds of the invention can be prepared as provided in Scheme 1. The skilled artisan will recognize that the invention compounds can be prepared from chiral starting materials or via racemic synthesis, followed by chiral separation, to isolate the enantiomers.

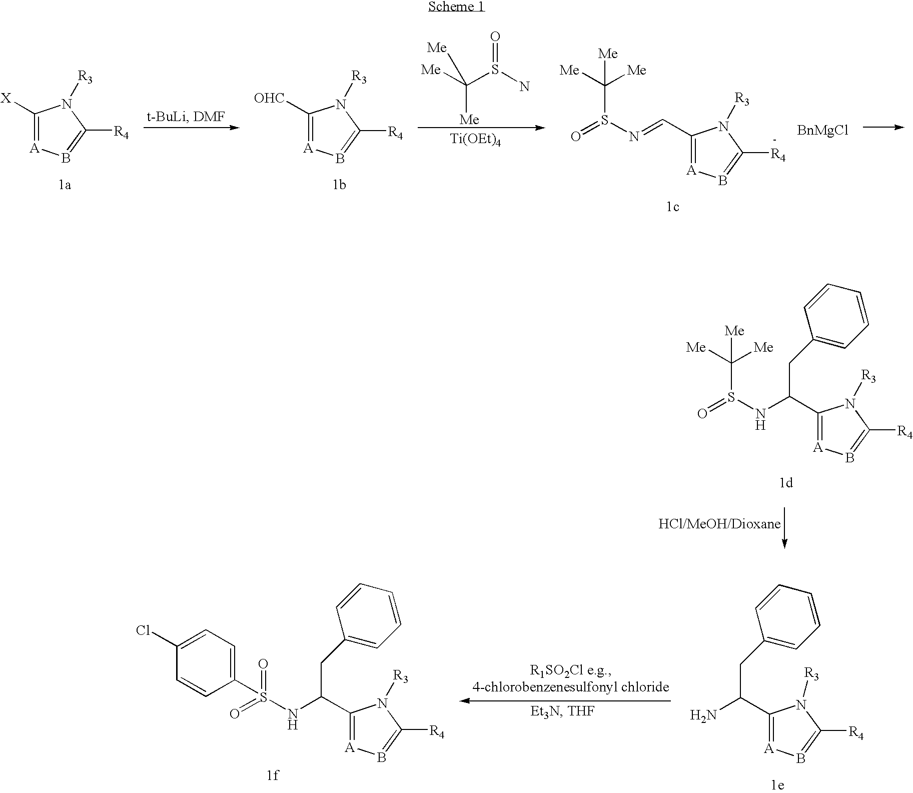

### Pharmaceutical Formulations

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. Examples of such acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, painoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred, although other salts are also useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

In order to use a compound of the formula (1) or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methylcellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Wherever formula I is used herein, formulae Ia, Ib and Ic can also be used.

According to a further aspect of the present invention there is also provided a compound of formula I or a pharmaceutically acceptable salt, prodrug, or solvate thereof in association with a pharmaceutically acceptable carrier, diluent, or excipient.

We have found that the compounds defined in the present invention, or a pharmaceutically acceptable salt thereof, are effective anti-cancer agents which property is believed to arise from their Edg-1 antagonistic properties. Accordingly the compounds of the present invention are expected to be useful in the treatment of diseases or medical conditions mediated alone or in part by Edg-1, i.e. the compounds may be used to produce an Edg-1 antagonistic effect in a warm-blooded animal such as man in need of such treatment.

What is also provided is a method of treating a disease or condition mediated by Edg-1 which comprises administering to a patient in need of such treatment a compound of formula I or a pharmaceutically acceptable salt, prodrug, or solvate thereof.

Thus the compounds of the present invention provide a method for treating cancer characterized by the antagonistic effect of Edg-1, i.e. the compounds may be used to produce an anti-cancer effect mediated alone or in part by the antagonistic effect of Edg-1.

Thus the compounds of the present invention provide a method for treating a variety of angiogenesis-related diseases that may be characterized by any abnormal, undesirable or pathological angiogenesis, for example tumor-related angiogenesis. The compounds may be used to produce an anti-cancer effect mediated alone or in part by antagonism of Edg-1. Such a compound of the invention is expected to possess a wide range of activity in angiogenesis-related diseases including, but not limited to, non-solid tumors such as leukemia, multiple myeloma, hematologic malignancies or lymphoma, and also solid tumors and their metastases such as melanoma, non-small cell lung cancer, glioma, hepatocellular (liver) carcinoma, glioblastoma, carcinoma of the thyroid, bile duct, bone, gastric, brain/CNS, head and neck, hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid tumors. Thus according to this aspect of the invention there is provided the use of a compound of the formula I or a pharmaceutically acceptable salt thereof in the manufacture of a medicament for use in the treatment of angiogenesis-related diseases including, but not limited to, non-solid tumors such as leukemia, multiple myeloma, hematologic malignancies or lymphoma, and also solid tumors and their metastases such as melanoma, non-small cell lung cancer, glioma, hepatocellular carcinoma, glioblastoma, carcinoma of the thyroid, bile duct, bone, gastric, brain/CNS, head and neck, hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid tumors.

In another embodiment the invention is directed to a method of treating of angiogenesis-related diseases including non-solid tumors, solid tumors and their metastases, non-small cell lung cancer, glioma, hepatocellular (liver) carcinoma, glioblastoma, carcinoma of the thyroid, bile duct, bone, gastric, brain/CNS, head and neck, hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid tumors, in a warm-blooded animal in need of such treatment which comprises administering to said animal an effective amount of a compound of formula I or a pharmaceutically acceptable salt thereof.

Excessive vascular growth also contributes to numerous non-neoplastic disorders for which the compounds of the invention may be useful in treating. These non-neoplastic angiogenesis-related diseases include: atherosclerosis, haemangioma, haemangioendothelioma, angiofibroma, vascular malformations (e.g. Hereditary Hemorrhagic Teleangiectasia (HHT), or Osler-Weber syndrome), warts, pyogenic granulomas, excessive hair growth, Kaposis' sarcoma, scar keloids, allergic oedema, psoriasis, dysfunctional uterine bleeding, follicular cysts, ovarian hyperstimulation, endometriosis, respiratory distress, ascites, peritoneal sclerosis in dialysis patients, adhesion formation result from abdominal surgery, obesity, rheumatoid arthritis, synovitis, osteomyelitis, pannus growth, osteophyte, hemophilia joints, inflammatory and infectious processes (e.g. hepatitis, pneumonia, glomerulonephritis), asthma, nasal polyps, liver regeneration, pulmonary hypertension, retinopathy of prematurity, diabetic retinopathy, age-related macular degeneration, leukomalacia, neovascular glaucoma, corneal graft neovascularization, trachoma, thyroiditis, thyroid enlargement, and lymphoproliferative disorders.

Thus according to this aspect of the invention there is provided a compound of the formula I or a pharmaceutically acceptable salt thereof, as defined hereinbefore for use as a medicament.

According to a further aspect of the invention there is provided the use of a compound of the formula I or a pharmaceutically acceptable salt thereof, as defined hereinbefore in the manufacture of a medicament for use in the production of a Edg-1 antagonistic effect in a warm-blooded animal such as man.

According to this aspect of the invention there is provided the use of a compound of the formula I or a pharmaceutically acceptable salt thereof, as defined hereinbefore in the manufacture of a medicament for use in the production of an anti-cancer effect in a warm-blooded animal such as man.

According to a further feature of the invention, there is provided a compound of the formula I or a pharmaceutically acceptable salt thereof, as defined herein before in the manufacture of a medicament for use in the treatment of pathologically angiogenic diseases, thrombosis, cardiac infarction, coronary heart diseases, arteriosclerosis, tumors, osteoporosis, inflammations or infections in a warm-blooded animal such as man.

According to a further feature of this aspect of the invention there is provided a method for producing a Edg-1 antagonistic effect in a warm-blooded animal, such as man, in need of such treatment which comprises administering to said animal an effective amount of a compound of formula I or a pharmaceutically acceptable salt thereof, as defined above.

According to a further feature of this aspect of the invention there is provided a method for producing an anti-cancer effect in a warm-blooded animal, such as man, in need of such treatment which comprises administering to said animal an effective amount of a compound of formula I or a pharmaceutically acceptable salt thereof, as defined above.

According to an additional feature of this aspect of the invention there is provided a method of treating pathologically angiogenic diseases, thrombosis, cardiac infarction, coronary heart diseases, arteriosclerosis, tumors, osteoporosis, inflammations or infections, in a warm-blooded animal, such as man, in need of such treatment which comprises administering to said animal an effective amount of a compound of formula I or a pharmaceutically acceptable salt thereof as defined herein before.

In a further aspect of the invention there is provided a pharmaceutical composition which comprises a compound of the formula I or a pharmaceutically acceptable salt thereof, as defined herein before in association with a pharmaceutically-acceptable diluent or carrier for use in the production of a Edg-1 antagonistic effect in a warm-blooded animal such as man.

In a further aspect of the invention there is provided a pharmaceutical composition which comprises a compound of the formula I or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula I or a pharmaceutically acceptable salt thereof, as defined herein before in association with a pharmaceutically-acceptable diluent or carrier for use in the treatment of pathologically angiogenic diseases, thrombosis, cardiac infarction, coronary heart diseases, arteriosclerosis, tumors, osteoporosis, inflammations or infections in a warm-blooded animal such as man.

What is also provided is a compound of formula I or a pharmaceutically acceptable salt, prodrug, or solvate thereof, which is an Edg-1 antagonist useful for controlling pathologically angiogenic diseases, thrombosis, cardiac infarction, coronary heart diseases, arteriosclerosis, tumors, osteoporosis, inflammations or infections.

### Combinations

The anti-cancer treatment defined herein may be applied as a sole therapy or may involve, in addition to the compound of the invention, conventional surgery or radiotherapy or chemotherapy. Such chemotherapy may include one or more of the following categories of anti-tumour agents:
1. antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
2. cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
3. agents which inhibit cancer cell invasion (for example metalloproteinase inhibitors like marimastat and inhibitors of urokinase plasminogen activator receptor function);
4. inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
5. antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
6. vascular damaging agents such as Combretastatin A4 and compounds disclosed in International Patent Applications WO 99/02166, WO 00/40529, WO 00/41669, WO 01/92224, WO 02/04434 and WO 02/08213;
7. antisense therapies, for example those which are directed to the targets listed above, such as ISIS 2503, an anti-ras antisense;
8. gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
9. immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

### Biological Activity

The following assay can be used to measure the effects of the compounds of the present invention as Edg-1 antagonists.

### A. In Vitro Cell Based Receptor Activation Assay-Transfluor Assay

This cell-based assay was designed to assess the ability of small molecule antagonists to inhibit activation of the GPCR S1P1 in the presence of its cognate ligand S1P. The assay used technology initially developed by Norak Biosciences (Xsira Pharmaceutical) and presently owned by Molecular Devices. A human osteogenic sarcoma (U2OS) cell line overexpressing the EDG-1/S1P1) receptor as well as a beta-arrestin/green fluorescent protein (GFP) construct hereafter termed EDG-1 Transfluor U2OS WT Clone #37 was employed.

Using a high content screening approach (Cellomics Arrayscan), receptor activity was measured by assessment of the relocalization of beta-arrestin GFP in response to stimulation of EDG-1 by S1P. Specifically, EDG-1 Transfluor U2OS WT Clone #37 cells were plated at a density of 6250 cells in 40 uL medium per well in 384 well plastic bottomed inicrotiter plates (BD Falcon) and incubated overnight at 37° C./5% CO₂. Prior to screening, compounds were dissolved in 100% dimethyl sulfoxide (DMSO) to a final stock concentration of 10 mM. Compounds were then serially diluted at 30× final concentration in EDG-1 Transfluor cell growth medium containing 30% DMSO using the Tecan Genesis instrument. These 30× plates were then diluted to 6× final concentration with EDG-1 Transfluor growth medium just prior to dosing. Cells were then dosed with 10 uL per well of 6× compound dilutions or 6% DMSO and pre-incubated for 15 minutes at room temperature. Cell plates were dosed with 10 uL per well 6× S1P EDG-1 Transfluor growth medium, then incubated for 45 minutes at 37° C./5% CO₂. Final concentration in the well of DMSO was 1%, compound was 1× (3-fold, 9 point IC50 dilutions starting at 100 uM final concentration), and either 375 mM or 750 nM S1P ligand. Cell plates were then fixed by adding 50 uL per well of 5% formaldehyde in 1× Dulbecco's phosphate buffered saline (DPBS) directly and incubating for 30 minutes at room temperature in darkness. Fixative was removed and replaced with 50 uL per well of 1×DPBS, after which cells were stained with 10 ug/mL final concentration of Hoechst 33342 (Molecular Probes) for 15 minutes at room temperature in darkness. Stain was then removed from the plates and replaced with 50 uL per well of 1×DPBS using the BioTek ExL405 plate washer. Plates were then sealed and analysed on the Cellomics Arrayscan using the GPCR signalling algorithm. EC₅₀ values were then calculated using IDBS ActivityBase software.

In this assay, compounds of the invention exhibit EC₅₀ values<100 μM; e.g., the compound of Example 1 had an EC₅₀ of 2.12 uM.

### EXPERIMENTAL SECTION

The invention will now be illustrated in the following Examples in which, generally:

(i) operations were carried out at ambient temperature, i.e. in the range 17 to 25° C. and under an atmosphere of an inert gas such as nitrogen or argon unless otherwise stated;

(ii) in general, the course of reactions was followed by thin layer chromatography

(TLC) and/or analytical high pressure liquid chromatography (HPLC); the reaction times that are given are not necessarily the minimum attainable;

(iii) when necessary, organic solutions were dried over anhydrous magnesium sulphate, work-up procedures were carried out using traditional layer separating techniques or an ALLEXIS (MTM) automated liquid handler, evaporations were carried out either by rotary evaporation in vacuo or in a Genevac HT-4/EZ-2.

(iv) yields, where present, are not necessarily the maximum attainable, and when necessary, reactions were repeated if a larger amount of the reaction product was required;

(v) in general, the structures of the end-products of the Formula I were confirmed by nuclear magnetic resonance (NMR) and/or mass spectral techniques; electrospray mass spectral data were obtained using a Waters ZMD or Waters ZQ LC/mass spectrometer acquiring both positive and negative ion data, generally, only ions relating to the parent structure are reported; proton NMR chemical shift values were measured on the delta scale using either a Bruker Spectrospin DPX300 spectrometer operating at a field strength of 300 MHz, a Bruker Dpx400 operating at 400 MHz or a Bruker Advance operating at 500 MHz. The following abbreviations have been used: s, singlet; d, doublet; t, triplet; q, quartet; m, multiplet; br, broad;

(vi) unless stated otherwise compounds containing an asymmetric carbon and/or sulphur atom were not resolved;

(vii) intermediates were not necessarily fully purified but their structures and purity were assessed by TLC, analytical HPLC, infra-red (IR) and/or NMR analysis;

(viii) unless otherwise stated, column chromatography (by the flash procedure) and medium pressure liquid chromatography (MPLC) were performed on Merck Kieselgel silica (Art. 9385);

(ix) preparative HPLC was performed on C18 reversed-phase silica, for example on a Waters ‘Xterra’ preparative reversed-phase column (5 microns silica, 19 mm diameter, 100 mm length) using decreasingly polar mixtures as eluent, for example decreasingly polar mixtures of water (containing 1% acetic acid or 1% aqueous ammonium hydroxide (d=0.88)) and acetonitrile;

(x) the following analytical HPLC methods were used; in general, reversed-phase silica was used with a flow rate of about 1 ml per minute and detection was by Electrospray Mass Spectrometry and by UV absorbance at a wavelength of 254 nm; for each method Solvent A was water and Solvent B was acetonitrile; the following columns and solvent mixtures were used:—

Preparative HPLC was performed on C18 reversed-phase silica, on a Phenominex “Gemini” preparative reversed-phase column (5 microns silica, 110A, 21.1 mm diameter, 100 mm length) using decreasingly polar mixtures as eluent, for example decreasingly polar mixtures of water (containing 0.1% formic acid or 0.1% ammonia) as solvent A and acetonitrile as solvent B; either of the following preparative HPLC methods were used:

Method A: a solvent gradient over 9.5 minutes, at 25 mls per minute, from a 85:15 mixture of solvents A and B respectively to a 5:95 mixture of solvents A and B.

Method B: a solvent gradient over 9.5 minutes, at 25 mls per minute, from a 60:40 mixture of solvents A and B respectively to a 5:95 mixture of solvents A and B.

(xi) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;

(xii) the following abbreviations have been used:—
DMF N,N-dimethylformamide
THF tetrahydrofuran
DCM Dichloromethane

### EXPERIMENTAL

### Example 1

### 4-Chloro-N-[1-(1,5-dimethyl-1H-imidazol-2-yl)-2-phenylethyl]benzenesulfonamide

### 1,5-dimethyl-1H-imidazole-2-carbaldehyde

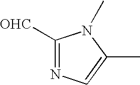

To a solution of 2-bromo-1,5-dimethyl-1H-imidazole (500 mg, 2.8 mmol) in THF (14 mL) was added t-BuLi (3.36 mL, 1.7 M in hexane) at −78° C. The reaction mixture was stirred for 45 min at this temperature before DMF (1.10 mL, 14.3 mmol) was added. The resulting solution was quenched with water then extracted with chloroform (2×20 mL). The combined organic layers were dried over Na₂SO₄ and concentrated to yield crude product as yellow solid, which was used directly in next step. M/Z=124.

### N-[(1E)-(1,5-dimethyl-1H-imidazol-2-yl)methylene]-2-methylpropane-2-sulfinamide

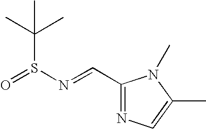

A solution of 1,5-dimethyl-1H-imidazole-2-carbaldehyde (prepared above) in THF was treated with 2-methylpropane-2-sulfinamide (518 mg, 4.28 mmol) and titanium ethoxide (2.35 mL, 11.2 mmol). The resulting solution was stirred for 48 h at room temperature. The reaction mixture was poured into brine (20 mL) slowly and extracted with chloroform (2×30 mL). The combined organic layers were dried over Na₂SO₄ and concentrated to yield crude product as yellow solid, which was used directly in next step. M/Z=227.

### N-[1-(1,5-dimethyl-1H-imidazol-2-yl)-2-phenylethyl]-2-methylpropane-2-sulfinamide

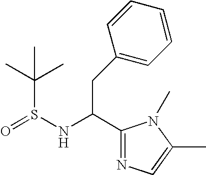

A solution of N-[(1E)-(1,5-dimethyl-1H-imindazol-2-yl)methylene]-2-methylpropane-2-sulfinamide (prepared above) in DCM was treated with benzyl magnesium chloride (5.2 mL, 10.4 mmol) at −78° C. The resulting solution was stirred for overnight and slowly warmed to room temperature. The reaction mixture was poured into a saturated solution of ammonia chloride (20 mL) slowly and extracted with DCM (2×30 mL). The combined organic layers were dried over Na₂SO₄ and concentrated to yield crude product as yellow solid, which was used directly in next step. M/Z=319

### 1-(1,5-dimethyl-1H-imidazol-2-yl)-2-phenylethanamine

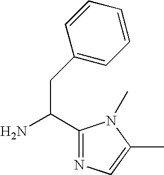

A solution of N-[1-(1,5-dimethyl-1H-imidazol-2-yl)-2-phenylethyl]-2-methylpropane-2-sulfinamide (prepared above) in MeOH (6 mL) was treated with hydrochloride acid (6 mL, 4M). The resulting solution was stirred for overnight. The reaction mixture was concentrated to yield crude product as viscous glue. To this material was added a solvents of MeOH/Et₂O (V/V=1:3, around 10 mL). The desired product was precipitated from solution as a white solid. M/Z=215.

### 4-chloro-N-[1-(1,5-dimethyl-1H-imidazol-2-yl)-2-phenylethyl]benzenesulfonamide

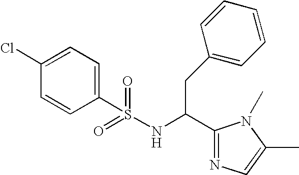

A solution of 1-(1,5-dimethyl-1H-imidazol-2-yl)-2-phenylethanamine (70 mg, 0.28 mmol) in THF (2 mL) was treated with 4-chlorobenzenesulfonyl chloride (51.2 mg, 0.28 mmol). The resulting solution was stirred for 0.5 h. The reaction mixture was concentrated to yield crude product as viscous glue, which was purified on reverse phase HPLC to yield the title compound, 4-chloro-N-[1-(1,5-dimethyl-1H-imidazol-2-yl)-2-phenylethyl]benzenesulfonamide, 45 mg). M/Z 389. ¹H NMR (300 MHz, CD₃OD) δ 7.56 (2H, s), 7.40 (2H, d), 7.10 (4H, m), 6.93 (2H, m), 4.8 (1H, m), 3.42 (3H, s), 3.15 (2H, m), 2.11 (3H, s).

What is claimed is:

1. A compound of formula I 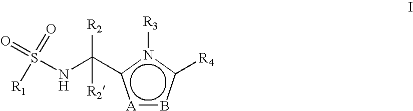
or a pharmaceutically acceptable salt thereof, wherein:
A and B are each independently N or CR_{b}, provided that A and B are not both N;
Rₐ independently at each occurrence is H, (C₁-C₆)alkyl, C(O)—(C₁-C₆)alkyl, C(O)—NR′R″, or CO₂(C₁-C₆)alkyl;
R_{b} at each occurrence is H, halo, (C₁-C₆)alkyl, cyano, —C(O)—(C₁-C₆)alkyl, —CO₂(C₁-C₆)alkyl, or C(O)—NR′R″, wherein R′ and R″ are each independently at each occurrence H, (C₁-C₆)alkyl, or X—R_{c}; —CO₂H, or —SO₂NHR;
R₁ is aryl, heteroaryl, (C₁-C₆)alkyl, aralkyl, heterocycloalkyl, or heteroaralkyl;
R₂ and R₂ are each independently H, (C₁-C₆)alkyl, aryl, heteroaryl, aralkyl, or heteroaralkyl, or taken together with the carbon to which they are attached from C═O;
R₃ is (C₁-C₆)alkyl, (C₃-C₆)cycloalkyl(C₁-C₆)alkyl, heterocycloalkyl, aralkyl, (C₂-C₆)alkenyl, (C₂-C₆)alkynyl, heteroaralkyl, or X—R_{c};
R₄ is (C₁-C₆)alkyl, (C₃-C₆)cycloalkyl(C₁-C₆)alkyl, heterocycloalkyl, aralkyl (C₁-C₆)alkenyl, (C₂-C₆)alkenyl, heteroaralkyl, or X—R_{c};
X is S, O, or NR_{d};
R_{c} is H or (C₁-C₆)alkyl;
R_{d} is H, (C₁-C₆)alkyl, aryl, heteroaryl, heterocyclo, (C₂-C₆)alkenyl, (C₂-C₆)alkynyl, aralkyl, heteroaralkyl, (C₃-C₆)cycloalkyl(C₁-C₆)alkyl, heterocycloalkyl(C₁-C₆)alkyl, acyl, acyloxy, acylamino, (C₁-C₆)alkoxycarbonyl(C₁-C₆)alkyl, or cyano; and
each R₁, R₂, R_{2′}, R₃, Rₐ, R_{b}, R_{c}, and R_{d} may be optionally substituted on carbon by azido, halo, nitro, cyano, hydroxy, trifluoromethoxy, NR′R″, —CO₂H, C(O)—(C₁-C₆)alkyl, —CO₂(C₁-C₆)alkyl, —C(O)—NR′R″, S(C₁-C₆), SOₚ(C₁-C₆)alkyl, SOₚNH(C₁-C₆)alkyl, SOₚNR′R″ (C₂-C₆)alkenyl, (C₂-C₆)alkynyl, or (C₁-C₆)alkoxy, wherein R′ and R″ are each independently hydrogen, (C₁-C₆)alkyl, (C₃-C₆)cycloalkyl, (C₃-C₆)cycloalkyl(C₁-C₆)alkyl, or aryl.

2. A compound according to claim 1 or pharmaceutically acceptable salt thereof wherein A is N.

3. A compound according to claim 1 or pharmaceutically acceptable salt thereof wherein R₁ is aryl optionally substituted by halo.

4. A compound according to claim 1 or pharmaceutically acceptable salt thereof wherein
A is N;
B is CR_{b};
R_{b} is selected from H, halo, (C₁-C₆)alkyl, cyano, —C(O)—(C₁-C₆)alkyl, —CO₂(C₁-C₆)alkyl, C(O)—NR′R″, wherein R′ and R″ are each independently at each occurrence H, (C₁-C₆)alkyl, or X—R_{c}; —CO₂H, or —SO₂NHR;
R₁ is aryl wherein aryl may be optionally substituted on carbon by azido, halo, nitro, cyano, hydroxy, trifluoromethoxy, NR′R″, —CO₂H, C(O)—(C₁-C₆)alkyl, —CO₂(C₁-C₆)alkyl, —C(O)—NR′R″, S(C₁-C₆), SOₚ(C₁-C₆)alkyl, SOₚNH(C₁-C₆)alkyl, SOₚNR′R″ (C₂-C₆)alkenyl, (C₂-C₆)alkynyl, or (C₁-C₆)alkoxy, wherein R′ and R″ are each independently hydrogen, (C₁-C₆)alkyl, (C₃-C₆)cycloalkyl, (C₃-C₆)cycloalkyl(C₁-C₆)alkyl, or aryl;
R₂ is selected from H and aralkyl;
R₂ is selected from H and aralkyl;
R₃ is (C₁-C₆)alkyl; and
R₄ is (C₁-C₆)alkyl.

5. A compound according to claim 1 or pharmaceutically acceptable salt thereof which is 4-chloro-N-[1-(1,5-dimethyl-1H-imidazol-2-yl)-2-phenylethyl]benzenesulfonamide.

6. A pharmaceutical composition which comprises a compound of the formula I, or a pharmaceutically acceptable salt thereof, as claimed in claim 1, in association with a pharmaceutically-acceptable carrier, diluent or excipient.

7. (canceled)

8. (canceled)

9. (canceled)

10. (canceled)

11. A method for producing an Edg-1 antagonistic effect in a warm-blooded animal, which comprises administering to said animal an effective amount of a compound of formula I, or a pharmaceutically acceptable salt thereof, as claimed in claim 1.

12. A method for producing an anti-cancer effect in a warm-blooded animal, which comprises administering to said animal an effective amount of a compound of formula I, or a pharmaceutically acceptable salt thereof, as claimed in claim 1.

13. A method of treating of angiogenesis-related diseases including non-solid tumors, solid tumors and their metastases, non-small cell lung cancer, glioma, hepatocellular (liver) carcinoma, glioblastoma, carcinoma of the thyroid, bile duct, bone, gastric, brain/CNS, head and neck, hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid tumors, in a warm-blooded animal in need of such treatment which comprises administering to said animal an effective amount of a compound of formula I or a pharmaceutically acceptable salt thereof, as claimed in claim 1.

14. (canceled)

15. (canceled)
